(19) United States
(12) Patent Application Publication
(10) Pub. No.: US 2010/0055775 A1
(43) Pub. Date: Mar. 4, 2010

(54) ORGANIC MATERIAL DIGESTER

(76) Inventor: Pearse O'Kane, Dublin (IE)

Correspondence Address: HOLLAND & KNIGHT LLP, 10 ST. JAMES AVENUE, BOSTON, MA, 02116-3889, US

(21) Appl. No.: 11/720,800

(22) PCT Filed: Dec. 2, 2005
(86) PCT No.: PCT/IE05/00137

§ 371 Date: Nov. 18, 2009

(30) Foreign Application Priority Data
Dec. 2, 2004 (IE) S2004/0808
Nov. 2, 2005 (IE) 2005/0727

(51) Int. Cl. C12M 1/10

(52) U.S. Cl. 435/290.3

(57) ABSTRACT

An organic material digester for the treatment of waste material which includes organic waste has an elongate rotating drum through which the waste is delivered between an inlet and outlet of the drum. Mechanical and biological activity within the drum converts the organic waste to a compost. A number of baffle plates sub-divide the drum into treatment compartments arranged in series between the inlet and the outlet. A port in each baffle plate offset from the centre of the baffle plate, allows through passage of material from one compartment to the next. Each baffle plate is secured to the drum by attachment means such as a number of spaced-apart radial rods which extend radially outwardly from the baffle plate through a side wall of the drum to which they are secured. Faces of the baffle plates are protected by a corrosion resistant material.

### BACKGROUND OF THE INVENTION

This invention relates to an organic material digester for the treatment of refuse and conversion of the organic portion of the refuse into a compost.

In U.S. Pat. No. 5,047,349 and U.S. Pat. No. 5,407,809 for example there are described systems and apparatus for converting organic waste material such as municipal waste into compost. The system shown in U.S. 5,047,349 operates in a batch treatment system, whilst the system of U.S. Pat. No. 5,407,809 is a continuous feed process. The content of these documents is included herein by reference. Each of these systems include a digester of the type comprising a cylindrical drum having an organic material inlet at one end and a treated material outlet at its other end, said cylindrical drum being rotatably mounted on a support for rotation about a central axis of the drum, means for rotating the drum on the support, the rotational axis of the drum being inclined downwardly between the inlet and the outlet of the drum to feed material between the inlet and the outlet, a plurality of spaced-apart baffles mounted within the drum between the inlet and the outlet to control the flow of material through the drum between the inlet and the outlet, each baffle having an opening for through passage of material. Within the, digester drum the baffles are typically welded to an inside face of the drum side-wall. During processing of the material in the drum a relatively corrosive environment is generated within the drum. In such a corrosive environment the welds can fail, leading to detachment of the baffles from the wall of the drum. In extreme cases the drum side-wall will crack.

The present invention is directed towards overcoming these problems.

### SUMMARY OF THE INVENTION

According to the invention there is provided a digester including, an elongate cylindrical drum, said drum being rotatably mounted on its side on a support for rotation about a central longitudinal axis of the drum, drum rotation means which is operable for rotation of the drum on the support, said drum having a raw material inlet and a treated material outlet remote from said raw material inlet, at least one baffle plate mounted within the drum between said raw material inlet and said treated material outlet for controlling the flow of material through the drum between the raw material inlet and the treated material outlet, said baffle plate or plates dividing an interior of the drum into two or more treatment compartments, the or each baffle plate having an opening for through passage of material from one compartment to the next adjacent compartment, the or each baffle plate being secured to the drum by attachment means which extends outwardly from the baffle plate through a cylindrical side wall of the drum.

In another embodiment the attachment means is secured to an exterior of the drum.

In one embodiment of the invention the drum has a support ring extending around an interior or an exterior of the drum in alignment with each baffle plate within the drum.

In another embodiment the attachment means is secured to the support ring.

In a further embodiment the attachment means includes a plurality of spaced-apart rods, each rod having an inner end and an outer end, said inner end being secured to the baffle plate and said outer end being secured to the drum.

In a preferred embodiment the inner end of each rod is secured at an outer circumferential edge of the baffle plate spaced inwardly from a front face and from a rear face of the baffle plate. In another embodiment the inner end of the rod locates in a complementary radial slot at an edge of the baffle plate.

In another embodiment the rods extend radially outwardly from the baffle plate through the drum side-wall.

In another embodiment an outer end of each rod engages in a complementary radial mounting hole in the support ring.

In a further embodiment at least the region where an outer edge of the baffle plate meets the drum side-wall is coated with a corrosion resistant material.

In a preferred embodiment each face of a baffle plate is coated with said corrosion resistant material.

Preferably the corrosion resistant material is an epoxy resin.

In another embodiment the baffle plate and associated rod connectors are of stainless steel material.

In another embodiment one or both faces of each baffle plate is protected by a sacrificial material. Preferably at least an upstream face of the baffle plate is protected by the sacrificial material.

Preferably the sacrificial material is made of hardwood, plastic or other shock and corrosion resistant material.

In another embodiment the inner end and the outer end of each rod are secured by welds or bolts.

In another embodiment the central longitudinal axis of the drum is inclined downwardly between the inlet and the outlet. The inclination may be between 0.5° and 3° to the horizontal preferably between 1° and 2.5°.

In a further embodiment each baffle plate has an opening for through passage of material which is located off-centre in the baffle plate.

Conveniently said opening has an elliptical shape. The opening may be arranged such that either a minor axis or a major axis of the elliptical opening lies along a radius of the baffle plate.

In a further embodiment an outer edge of the opening in the or each baffle plate is spaced inwardly from an inside face of the drum side wall.

In another embodiment a door is mounted at the opening in each baffle plate, said door being operable to open and dose said opening.

In another embodiment the drum rotation means for rotating the drum on the support includes a ring gear which extends around a circumference of the drum, a complementary pinion driveably engaged with the ring gear, and a drive motor connected through a reduction gearbox with the pinion.

In a further embodiment the drum rotation means for rotating the drum on the support comprises a tire which extends around a circumference of the drum and is fixed thereto, said tire resting on and supported by a pair of spaced-apart wheels which are rotatably mounted on a support frame, at least one of said wheels being a friction drive wheel connected to an associated drive motor operable to rotate the friction drive wheel on the support frame for rotation of the drum.

In another embodiment the drive motor is a hydraulic motor.

In a further embodiment two tires are mounted spaced-apart on the drum, each of said tires having associated drive means for rotation of the tire and hence the drum.

In a particularly preferred embodiment the cylindrical drum is protected internally by longitudinal stress bars fixed to an inside face of the drum and extending along the length of the drum, each of said stress bars projecting inwardly from the inside face of the drum. Ideally the stress bars are approximately four to five inches (100 mm-125 mm) high, about one inch (25 mm) thick and spaced about four to five inches (100 mm-125mm) apart. Channels formed between these stress bars fill with organic material when the drum is in use and greatly enhance the impact absorption as well as provide the biological medium required to accelerate the fermentation process of the organic material.

### BRIEF DESCRIPTION OF THE DRAWINGS

The invention will be more clearly understood by the following description of some embodiments thereof, given by way of example only, with reference to the accompanying drawings, in which;

FIG. 1 is a schematic illustration of a digester according to the invention in use in a waste processing system;

FIG. 2 is another schematic illustration of several digesters operating in the waste processing system;

FIG. 3 is a perspective view of a digester according to the invention;

FIG. 4 is a schematic cross sectional illustration of the digester;

FIG. 5 is an elevational view of another digester according to the invention;

FIG. 6 is a view similar to FIG. 5 of the digester of FIG. 5;

FIG. 7 is an end elevational view taken along the line VII-VII of FIG. 6;

FIG. 8 is an end elevational view taken along the line VIII-VIII of FIG. 6;

FIG. 9 is a sectional elevational view taken along the line IX-IX of FIG. 6;

FIG. 10 is a sectional elevational view taken along the line X-X of FIG. 6;

FIG. 11 is a detail sectional view showing portion of a cylindrical drum of the digester; and

FIG. 12 is a detail sectional view showing portion of a drum wall of the digester.

### DETAILED DESCRIPTION OF PREFERRED EMBODIMENTS

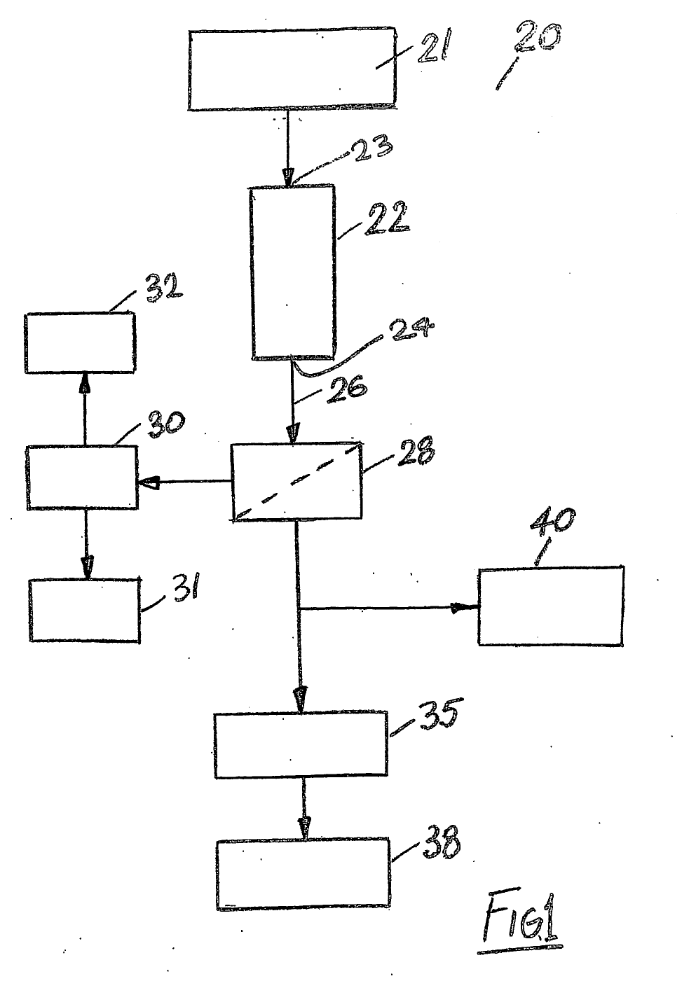

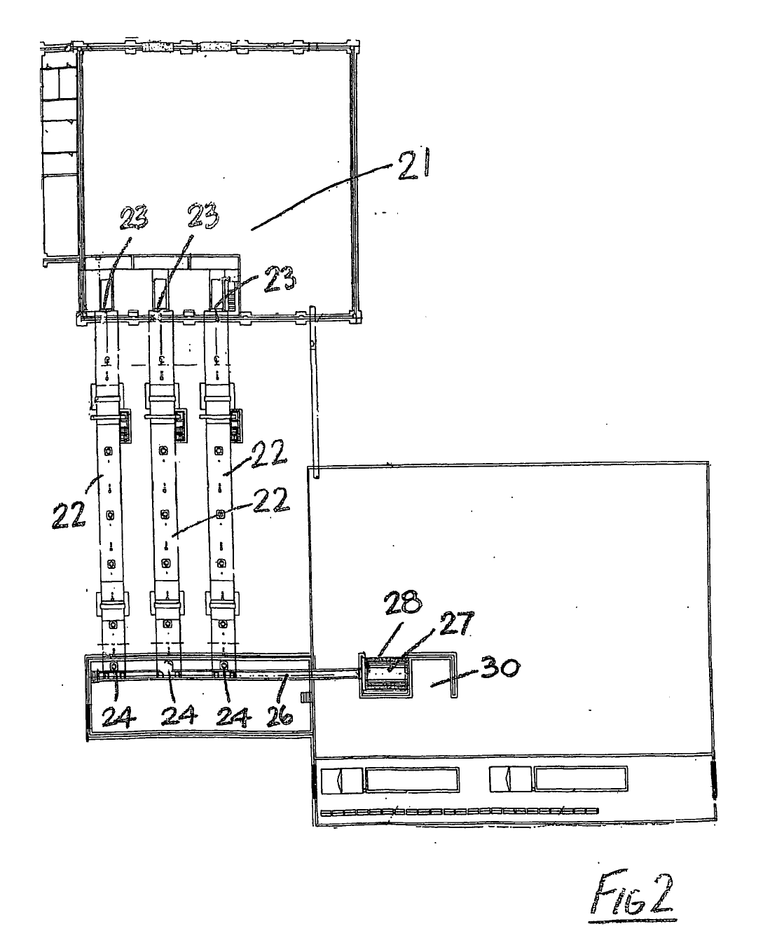

Referring to the drawings, and initially to FIGS. 1 and 2 thereof, there is illustrated a waste treatment system indicated generally by the reference numeral 20 which incorporates one or more digesters according to the invention. The system includes a waste collecting station 21 at which raw waste material such as municipal waste, household waste and the like is collected. This raw waste material is then fed to an inlet of a rotary organic material digester 22 according to the invention. The raw waste material is delivered through the rotary digester 22 in a controlled manner between an inlet 23 and an outlet 24 of the digester 22 for converting the organic waste material content of the raw waste material to a bio-fuel and/or compost.

The rotary organic material digester 22 includes a cylindrical drum having a raw material inlet 23 at one end and a treated material outlet 24 at the other end. The cylindrical drum is rotatably mounted on a support for rotation about a central axis of the drum. The rotational axis of the drum is inclined downwardly between the inlet 23 and the outlet 24 of the drum to gradually feed material between the inlet 23 and the outlet 24 of the drum as the drum is rotated. Various types of digester according to the invention are described more fully later.

FIG. 2 shows three of the digesters 22 arranged in parallel for treating the waste material. Treated material is discharged from an outlet 24 of each digester 22 onto a conveyor 26 which delivers the treated material to a hopper 27 feeding a screen 28. The screen 28 separates the organic from the inorganic (non-biodegradable) materials in the treated material. The sanitised inorganic material is delivered to a residual collection bay 30 and is subsequently sorted into recyclable material 31 and non-recyclable material 32, which is delivered to a landfill for disposal.

The organic material may be delivered from the screen 28 for use as a bio-fuel.

Typically the material is delivered to a drying station 35 in which the moisture content of the bio-fuel material is reduced by compression of the bio-fuel material and/or any other suitable drying method. The dried bio-fuel material can then be used as a bio-fuel in a generating boiler 38 for example, or as any fossil fuel alternative.

In addition or alternatively organic material may be delivered downstream of the screen 28 to a compost bay 40 for subsequent delivery for use as agricultural or horticultural compost. Further curing or maturing of the compost may be carried out before use of the compost.

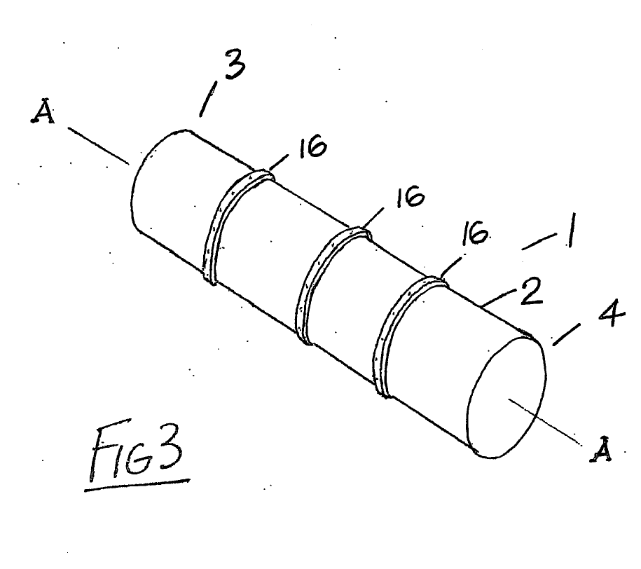

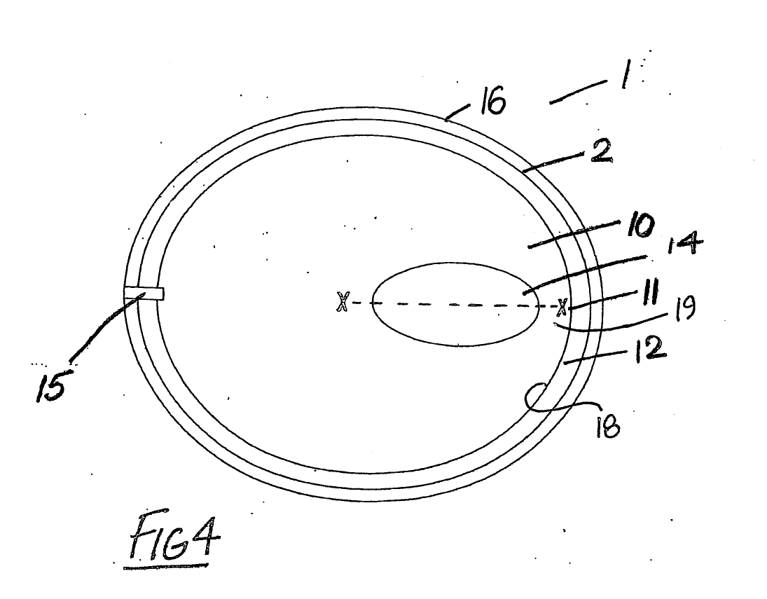

Referring now to FIGS. 3 and 4 of the drawings there is illustrated a digester according to the invention indicated generally by the reference numeral 1. The digester 1 has a cylindrical drum 2 which is rotatably mounted for rotation about a central longitudinal axis A of the drum 2. Drive means (not shown) is provided for rotating the drum 2 about said longitudinal axis A in an inclined position to deliver material between an inlet end 3 and an outlet end 4 of the drum for treatment as it passes through the drum 2.

A plurality of baffle plates 10 are mounted spaced-apart within the drum 2 between the inlet 3 and the outlet 4 of the drum 2 to separate the drum interior into a number of treatment compartments and to control the flow of material through the drum 2 between the inlet 3 and the outlet 4. Each baffle plate 10 is circular and is a close fit within the drum 2, an outer circumferential edge 11 of the baffle plate 10 abutting an inner face of a cylindrical side wall 12 of the drum 2. The baffle plate 10 is formed of stainless steel material and has a protective epoxy resin coating. Additional impact protection may also be provided as described later.

Each baffle plate 10 is mounted within the drum 2 substantially perpendicular to the longitudinal axis A of the drum 2. The baffle plates 10 subdivide the drum 2 into a number of treatment compartments arranged in series between the inlet 3 and outlet 4 of the drum 2. An opening or port 14 in each baffle plate 10 allows controlled through passage of material from one compartment to the next as the material travels between the inlet 3 and outlet 4 of the drum 2. The opening or port 14 is open, or if required fitted with a door (not shown) which allows complete segregation in the area or compartment between the baffles 10. In the embodiment shown in FIG. 4 the port 14 is generally elliptical in shape. The port 14 is located off-centre in the baffle plate 10 with a major axis X of the port 14 generally lying along a radius of the baffle plate 10. An outer edge 17 of the port 14 is spaced inwardly from an inside face 18 of the drum side wall 12 leaving a lip or step 19 which extends inwardly from the face 18 of the drum side wall 12.

Each baffle plate 10 is secured within the drum 2 by means of a plurality of circumferentially spaced-apart stainless steel rods 15, only one of which is illustrated in FIG. 4. Each rod 15 extends radially outwardly from the baffle plate 10 through an opening in the drum side-wall 12, engaging a stainless steel outer support ring 16 which extends around the drum 2 in alignment with the associated baffle plate 10. A plurality of spaced-apart radial holes are provided in the ring 16 for reception of the rods 15 which are welded or bolted to the ring 16 after welding to the baffle plate 10. The support ring 16 is a close fit about the drum 2 and is welded to an exterior of the drum 2. In an alternative arrangement the support ring 16 may be mounted inside the drum 2 instead of outside as shown.

The rods 15 form an attachment means which extends outwardly from the baffle plate 10 through the side wall 12 of the drum 2, anchoring the baffle plate 10 in the drum side wall 12.

A surface of each baffle plate 10 and the region where an outer edge of each baffle plate 10 engages the side-wall 12 of the drum 2 is coated with an epoxy coating. A suitable coating is the two component, solvent based epoxy coating FX-470 supplied by Fox Industries.

In use, the digester 1 is slowly rotated. Raw waste material is delivered to the inlet 3 of the digester 1 and travels through the digester 1 between the inlet 3 and the outlet 4. Within the digester 1 mechanical activity due to tumbling of the waste and biological activity due to aerobic bacteria converts the organic waste material content of the raw waste material into a bio-fuel or compost. Treated material is discharged from the outlet 4 of the drum 2. The treated material will include organic/biodegradable material and inorganic/non-biodegradable material. This can be separated as previously described.

It will be noted that with the baffle mounting arrangement of the invention all welded joints are protected against any corrosive environment within the digester drum 2. Further, the support ring 16 gives added reinforcement to the drum 2 at each baffle mounting location.

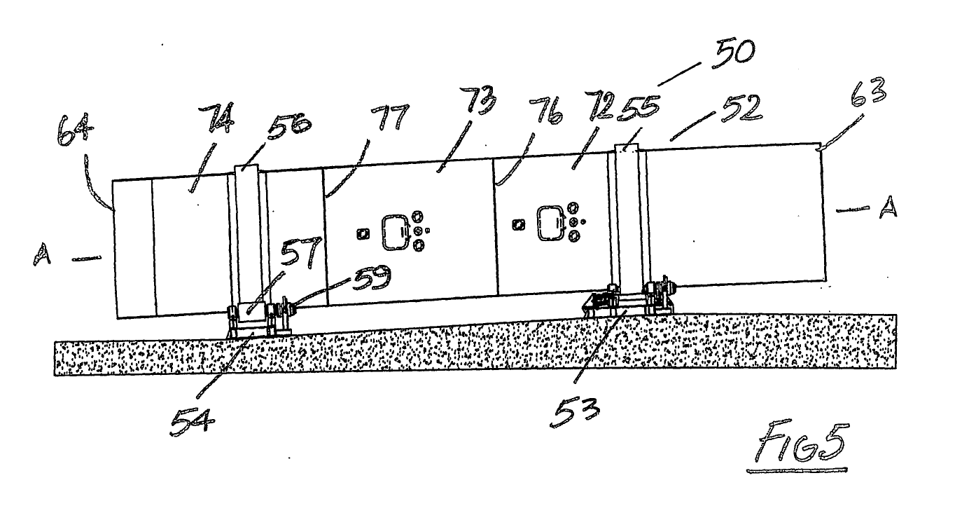

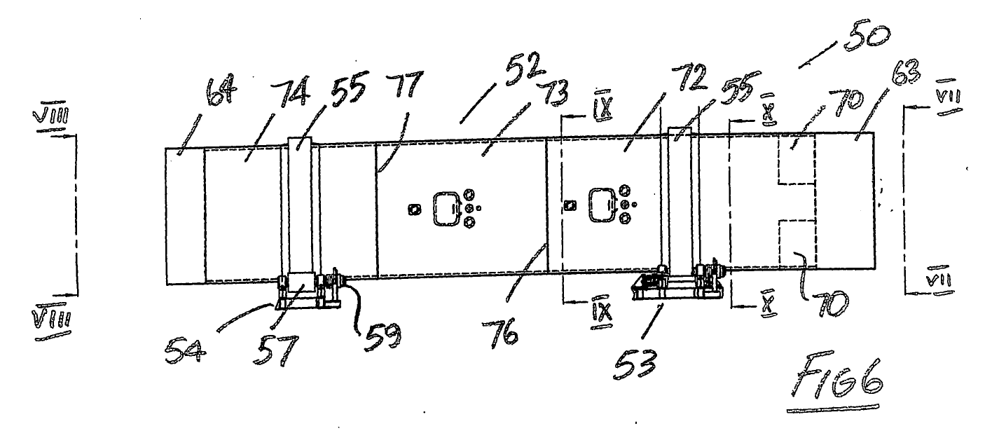

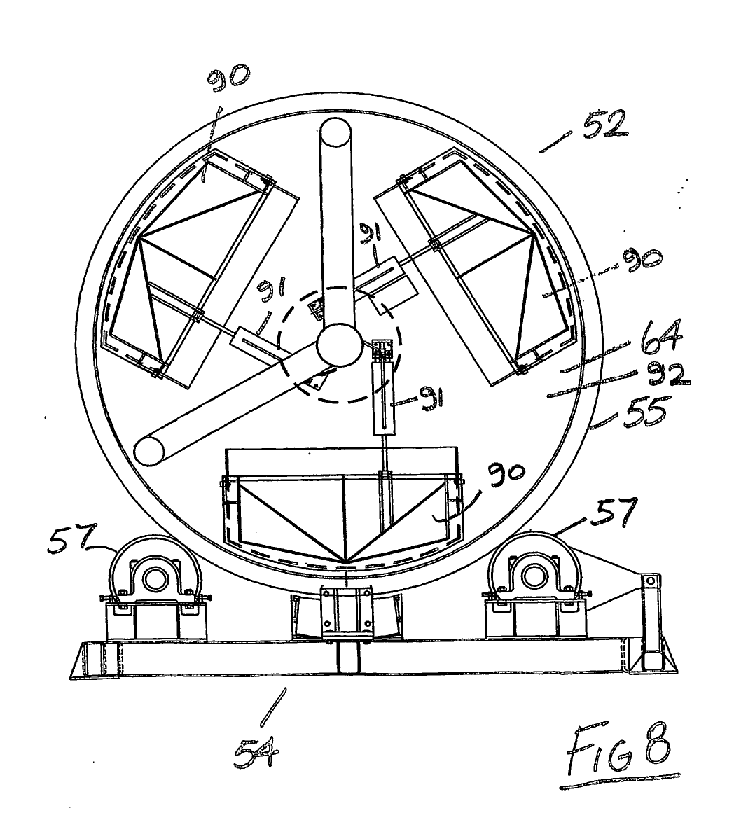

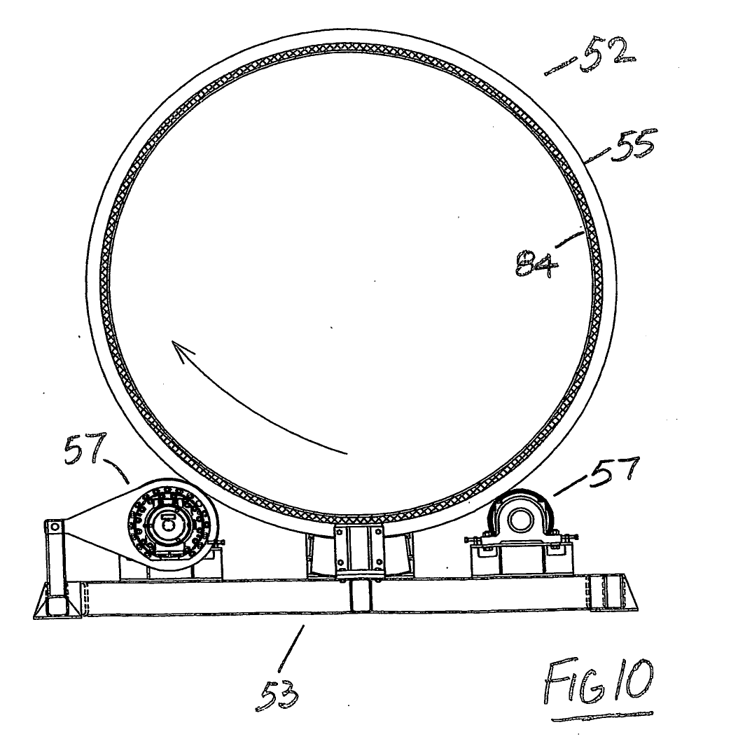

Referring to FIGS. 5 to 12 there is shown another digester according to the invention indicated generally by the reference numeral 50. The digester 50 comprises a cylindrical drum 52 which is rotatably mounted on a pair of spaced-apart support frames, namely a front support frame 53 and a rear support frame 54. Two spaced-apart metal tires 55, 56 extend around a circumference of the drum 52. Each tire 55, 56 is supported by a pair of spaced-apart trunnions or wheels 57 (best seen in FIGS. 8 and 10) which are rotatably mounted on the support frames 53, 54. At least one of the wheels 57 comprises a friction drive wheel which is connected to an associated drive motor 59 to rotate the friction drive wheel 57 on the support frame 53, 54 for rotation of the drum 52 on the support frames 53, 54. Preferably friction drive wheels 57 are provided at each support frame 53, 54. The drive motors 59 may conveniently be either hydraulic motors or electric motors. Any other arrangement for driving the friction drive wheels 57 or otherwise rotating the drum 52 may be provided as an alternative.

It will be noted that a central longitudinal axis A of the drum 52 about which the drum 52 rotates is inclined at an angle of about 10 to the horizontal. Thus the drum 52 is inclined downwardly between an inlet end 63 and outlet end 64 of the drum 52. This provides for gravity feed of material through the drum 52 between the inlet end 63 and outlet end 64 as the drum 52 is rotated.

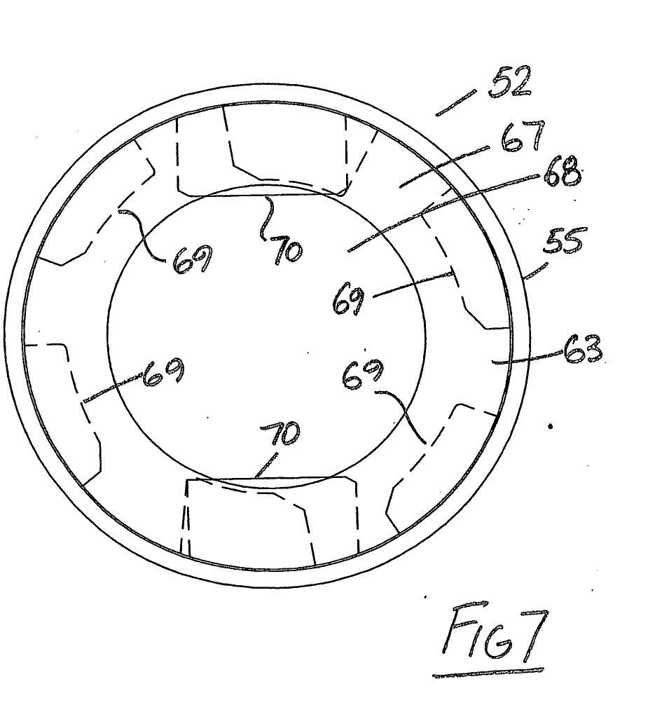

The inlet end 63 of the drum 52 can be seen in more detail in FIG. 7. An annular end plate 67 has a central circular inlet opening 68. A number of scoop plates 69 mounted on an inside face of the drum wall project inwardly from the drum wall. The scoop plates 69 feed waste material into transfer boxes 70 for delivery to an interior of the drum 52.

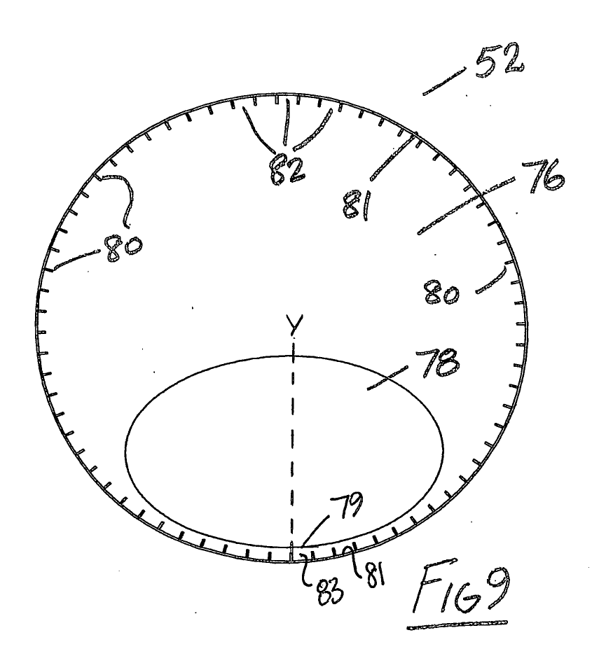

Referring to FIGS. 5 and 9, an interior of the drum 52 is divided into three compartments namely a first compartment 72 a second compartment 73 and a third compartment 74 by a pair of spaced-apart baffle plates 76, 77. As previously described these baffle plates 76, 77 control the flow of material through the drum 52 between the inlet 63 and outlet 64.

Each baffle plate 76, 77 is mounted within the drum 52 substantially perpendicular to the longitudinal axis A of the drum 52. The mounting is preferably as described previously for the baffle plate shown in FIG. 4. An opening or port 78 in each baffle plate 76, 77 allows through passage of material between adjacent compartments 72, 73, 74 separated by the baffle plate 76, 77. In this case the port 78 has an elliptical shape with a minor axis Y of the ellipse substantially coincident with a radius of the baffle plate 76, 77 or drum 52. As can be seen in FIG. 9 the port 78 is located offset from a centre of the drum 52. An outer edge 79 of the port 78 is spaced inwardly from an inside face 81 of the drum side, wall leaving a lip or step 83 which extends inwardly from the face 81 of the drum side wall.

Referring to FIG. 9 the drum 52 is protected internally by a plurality of spaced-apart longitudinal stress bars 80 which project radically inwardly from an inside face 81 of the drum 52. The stress bars 80 are fixed to the inside face 81 and extend along the length of the drum 52. The stress bars 80 are approximately four to five inches (100 mm-125 mm) high, about one inch (25 mm) thick and about four to five inches (100 mm-125 mm) apart. Channels 82 formed between these stress bars 80 fill with organic material when the drum 52 in use and greatly enhance the impact absorption as well as provide biological medium required to accelerate the fermentation process of the organic material.

Preferably each baffle plate 76, 77 is made from stainless steel or some other anti-corrosion material. In addition, each face of the baffle plate 76, 77 may be protected by a sacrificial material such as hardwood, plastic or other shock and corrosion resistant material. This prevents damage to the baffle plate 76, 77 during operation of the drum 52.

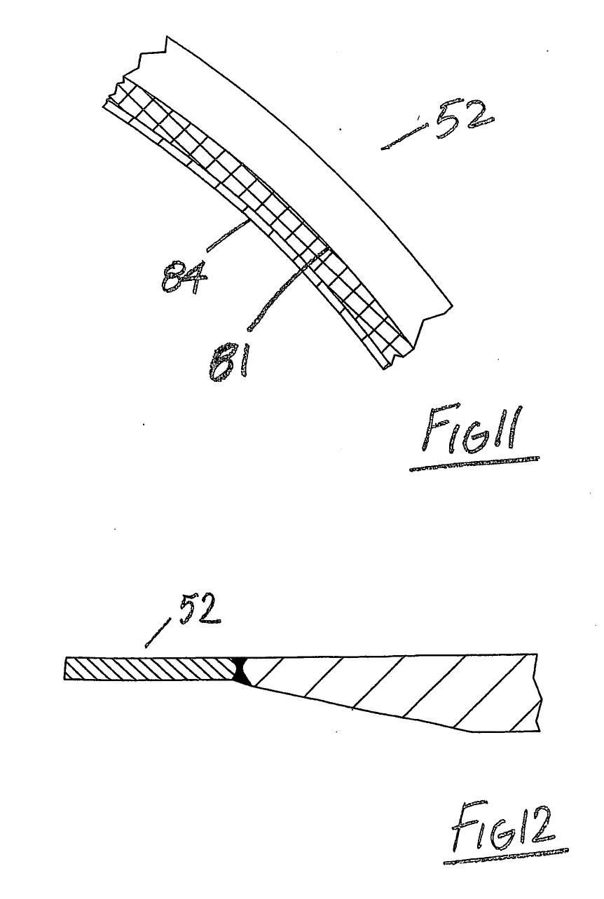

The inside face 81 of the drum 52 is coated with a 50 mm layer 84 of polyurethane to protect the drum side wall, as shown in FIGS. 10 and 11. An external coating of polyurethane foam is used to insulate the drum from variable weather conditions.

Referring to FIG. 8 the outlet end 64 of the drum 52 is shown. A number of discharge doors 90 operated by rams 91 are mounted on an end plate 92 of the drum 52. The doors 90 can be opened and closed by the rams 91 for controlling discharge of treated material from the drum 52.

FIG. 12 shows reinforcement of the drum side wall at the tire mounting locations.

It will be appreciated that the digester may have one or more compartments for the treatment of the waste material. Where a number of compartments are provided these may be provided as described by sub-dividing the drum into a number of compartments or indeed a number of drums, each comprising one compartment, may be arranged in series with material being passed from one drum to the next.

Instead of the rods anchoring each baffle plate in the drum side wall each baffle may have integral arms spaced around a periphery of the baffle plate and extending radially outwardly therefrom. If the drum is constructed in sections these arms locate between abutting edges of the drum sections, with appropriate cut-outs in said edges to receive the arms, before welding the drum sections together.

If desired the thickness of the wall of the drum in the region of each baffle may be increased for added reinforcement.

While in the embodiments described above the drum is inclined downwardly between the inlet and the outlet, in some cases the drum may be supported in a substantially horizontal orientation for rotation about a horizontal central longitudinal axis of the drum.

Also, means may be provided to force feed the waste material into the inlet of the drum—by means of a ram or inlet feed auger for example.

The invention is not limited to the embodiments hereinbefore described which may be varied in both construction and detail within the scope of the appended claims.

1-45. (canceled)

46. A digester including:
an elongate cylindrical drum,
said drum being rotatably mounted on its side on a support for rotation about a central longitudinal axis of the drum,
drum rotation means which is operable for rotation of the drum on the support,
said drum having a raw material inlet and a treated material outlet remote from said raw material inlet,
at least one baffle plate mounted within the drum between said raw material inlet and said treated material outlet for controlling the flow of material through the drum between the raw material inlet and the treated material outlet,
said baffle plate or plates dividing an interior of the drum into two or more treatment compartments,
the or each baffle plate having an opening for through passage of material from one compartment to the next adjacent compartment,
the or each baffle plate being secured to the drum by attachment means which extends outwardly from the baffle plate through a cylindrical side wall of the drum.

47. A digester as claimed in claim 46 wherein the attachment means is secured to an exterior of the drum.

48. A digester as claimed in claim 46 wherein the drum has a support ring extending around the drum in alignment with each baffle plate within the drum.

49. A digester as claimed in claim 48 wherein the support ring extends around an exterior of the drum.

50. A digester as claimed in claim 48 wherein the support ring extends around an interior face of the drum sidewall.

51. A digester as claimed claim 48 wherein the attachment means is secured to the support ring.

52. A digester as claimed in claim 46 wherein the attachment means includes a plurality of spaced-apart rods, each rod having an inner end and an outer end, said inner end being secured to the baffle plate and said outer end being secured to the drum.

53. A digester as claimed in claim 52 wherein the inner end of each rod is secured at an outer circumferential edge of the baffle plate spaced inwardly from a front face and from a rear face of the baffle plate.

54. A digester as claimed in claim 52 wherein the inner end of the rod locates in a complementary radial slot at an outer edge of the baffle plate.

55. A digester as claimed in claim 52 wherein the rods extend radially outwardly from the baffle plate through the drum side wall.

56. A digester as claimed in claim 55 wherein an outer end of each rod engages in a complementary radial mounting hole in the support ring.

57. A digester as claimed in claim 46 wherein at least an upstream face of each baffle plate is protected by a sacrificial material.

58. A digester as claimed in of claim 52 wherein the inner end and the outer end of each rod are secured by welds.

59. A digester as claimed in claim 46 wherein the opening in each baffle plate for through passage of material is located off-centre in the baffle plate.

60. A digester as claimed in claim 59 wherein said opening has an elliptical shape.

61. A digester as claimed in claim 60 wherein either a minor axis or a major axis of the elliptical opening lies along a radius of the baffle plate.

62. A digester as claimed in claim 46 wherein an outer edge of the opening in the or each baffle plate is spaced inwardly from an inside face of the drum side wall.

63. A digester including:
an elongate cylindrical drum,
said drum being rotatably mounted on its side on a support for rotation about a central longitudinal axis of the drum,
drum rotation means which is operable for rotation of the drum on the support,
said drum having a raw material inlet and a treated material outlet remote from said raw material inlet,
at least one baffle plate mounted within the drum between said raw material inlet and said treated material outlet for controlling the flow of material through the drum between the raw material inlet and the treated material outlet,
said baffle plate or plates dividing an interior of the drum into two or more treatment compartments,
the or each baffle plate having an opening for through passage of material from one compartment to the next adjacent compartment,
the or each baffle plate being secured to the drum by attachment means which extends outwardly from the baffle plate through a cylindrical side wall of the drum,
the opening in each baffle plate for through passage of material being located off-centre in the baffle plate.

64. A digester including:
an elongate cylindrical drum,
said drum being rotatably mounted on its side on a support for rotation about a central longitudinal axis of the drum,
drum rotation means which is operable for rotation of the drum on the support,
said drum having a raw material inlet and a treated material outlet remote from said raw material inlet,
at least one baffle plate mounted within the drum between said raw material inlet and said treated material outlet for controlling the flow of material through the drum between the raw material inlet and the treated material outlet,
said baffle plate or plates dividing an interior of the drum into two or more treatment compartments,
the or each baffle plate having an opening for through passage of material from one compartment to the next adjacent compartment,
said opening in the baffle plate being located off-centre in the baffle plate and an outer edge of the opening in the or each baffle plate being spaced inwardly from an inside face of a side wall of the drum.

65. A digester including:
an elongate cylindrical drum,
said drum being rotatably mounted on its side on a support for rotation about a central longitudinal axis of the drum, drum rotation means which is operable for rotation of the drum on the support,
said drum having a raw material inlet and a treated material outlet remote from said raw material inlet,
at least one baffle plate mounted within the drum between said raw material inlet and said treated material outlet for controlling the flow of material through the drum between the raw material inlet and the treated material outlet,
said baffle plate or plates dividing an interior of the drum into two or more treatment compartments,
the or each baffle plate having an opening for through passage of material from one compartment to the next adjacent compartment,
the or each baffle plate being secured to the drum by attachment means which extends outwardly from the baffle plate through a cylindrical side wall of the drum,
the central longitudinal axis of the drum being inclined downwardly between the inlet and the outlet of the drum to feed material between the inlet and the outlet,
the opening in each baffle plate for through passage of material being located off-centre in the baffle plate, said opening having an elliptical shape,
an outer edge of the opening in the or each baffle plate being spaced inwardly from an inside face of the drum side wall,
the drum rotation means for rotating the drum on the support including a tire which extends around a circumference of the drum and is fixed thereto, said tire resting on and supported by a pair of spaced-apart wheels which are rotatably mounted on a support frame, at least one of said wheels being a friction drive wheel connected to an associated drive motor which is operable to rotate the friction drive wheel on the support frame for rotation of the drum.
